(19) United States
(12) Patent Application Publication
(10) Pub. No.: US 2008/0051627 A1
(43) Pub. Date: Feb. 28, 2008

(54) ENDOSCOPIC INSERTION OF BALLOON SYSTEM AND METHOD

(75) Inventor: Gottumukkala RAJU, League City, TX (US)

Correspondence Address: LOCKE LORD BISSELL & LIDDELL LLP;ATTN: IP DOCKETING, 600 TRAVIS, SUITE 3400, HOUSTON, TX, 77002-3095, US

(73) Assignee: BOARD OF REGENTS, THE UNIVERSITY OF TEXAS SYSTEM, Austin, TX (US)

(21) Appl. No.: 11/844,495

(22) Filed: Aug. 24, 2007

Related U.S. Application Data
(60) Provisional application No. 60/823,674, filed on Aug. 28, 2006.

(51) Int. Cl. A61B 1/018; A61B 1/01

(52) U.S. Cl. 600/104; 600/114; 600/116

(57) ABSTRACT

The present disclosure provides a method and system to facilitate endoscopic insertion of a balloon into tissues, such as submucosal tissues, and optional resection of tissues elevated by the balloon when inflated. The system includes an endoscope with a delivery system for a detachable balloon having a seal. The balloon can be inserted under the tissue, such as a submucosal insertion, and inflated. The balloon can then be untethered from the delivery system and deployed submucosally while the endoscope is manipulated around the affected area and the affected tissue knife resected. The endoscope can be retracted from the area. The balloon can be made of biodegradable material that can dissolve or be absorbed over a period of time. The system and method can be applied to other areas and medical procedures that require insertion of a balloon for a period of time.

### CROSS REFERENCE TO RELATED APPLICATIONS

This application claims the benefit of U.S. Provisional Application Ser. No. 60/823,674, filed Aug. 28, 2006, which is incorporated herein by reference.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO APPENDIX

Not applicable.

### BACKGROUND

1. Field of the Invention

The invention relates to medical procedures. More specifically, the invention relates to the use of balloons inserted under tissues for enlargement and optional resection of the tissue.

2. Description of Related Art

Tissues, especially mucosal tissues, form an inner lining of the mouth, nasal passages, esophagus, gastrointestinal tract, and other body passages. These tissues are prone to polyps, lesions, tumors, or other types of protruding growths. Several technologies and methods have evolved to remove such growths. Laparoscopic surgery evolved in the 1990's and assumed an important role in the management or excision of cancerous segments of the bowel or polyp removal. The less invasive approach of laparoscopy reduced hospital time to a few days, and spared the patient a large abdominal incision in place of a few small cuts through which the laparoscopic devices were inserted. Even these procedures require repeat hospital visit for surgery, general anesthesia, two to three day hospitalization, and potential complications related to surgery and anesthesia.

However, additional advances in less invasive surgical procedures were needed and resulted in development of endoscopic surgery. Endoscopic surgery relies on oral or anal insertion for surgery that can be viewed as a major advance beyond laparoscopy in that it typically can be accomplished at the time of diagnosis, it does not typically require anesthesia, and it avoids the complications associated with body-piercing incision to gain access to the colon or gastric site for tissue removal. Endoscopic mucosal resection (EMR), which is widely practiced in Japan, is gaining acceptance in the rest of the world. However, current endoscopic methods to dissect such growths have restricted access and manipulation of instruments and therefore are cumbersome and time consuming. Typically, five to six hours is required to do the dissection, depending on the type of growth and its protrusion above the tissue surface. Although this is a standard method of treatment of gastrointestinal polyps, it is associated with substantial risk of complications.

The length of time depends on the growth size and the amount of protrusion above the tissue surface, with more time generally required for a growth closer to the tissue surface, that is, shorter in height. For illustration, FIG. 1 is a schematic cross sectional view of tissues with growths. A mucosal tissue 50 is the tissue typically prone to growths. A submucosal tissue 52 underlies the mucosal tissue, with a muscularis tissue 54 under the submucosal tissue, and a serosa membrane tissue 55 under the muscularis tissue. A growth 56A, such as a polyp, generally has a head that can be removed by a snare in a snare polypectomy procedure. The snare is positioned to encircle the polyp, then constricted below the head, and excised from the mucosal tissue. However, excision of a larger, flatter, or restricted access growth 56A, such as a lesion, can be problematic.

Typically, an endoscope will be guided to the location. The simplest procedure is when the growth is sufficiently protruding above the tissue and shaped appropriately, such as the growth 56A. A “snare” can be sent through the endoscope to encircle the growth and resect it from the surrounding tissue. A more complicated procedure occurs when the growth is not protruding much, if at all, above the tissue surface, such as growth 56B. Surgical removal with a knife guided through the endoscope of a minimally protruding growth is more complicated, because the possibility increases dramatically of causing hemorrhaging (up to about 6% of the cases) in the submucosal tissue or even perforating the tissue wall rather than resecting a tissue layer. This possibility is especially true in relatively thin walls, for example, three to five millimeters in thickness of some of the tissues frequently affected. Perforating the wall to expose other tissues and body cavities can lead to significant complications.

Three factors can make endoscopic resection of growths difficult, such as colonic polyps. These are size, configuration, and location. For instance, sessile polyps greater than two centimeters in diameter, depressed or occupying more than one-third of the wall circumference, extending over more than two folds, or wrapped around a fold in a clamshell fashion, can make polypectomy a challenge. In addition, sessile polyps located behind a fold, within a flexure, or in a tortuous segment of the colon (such as the sigmoid), present a particular challenge even to the skilled endoscopist.

One recent method of dissecting such growths is to inject a saline solution below the growth, that is, at a submucosal level and “swelling” the tissue. This technique, sometimes references as a “submucosal saline-epinephrine injection polypectomy” provides an increased safety margin when performing a polypectomy, either by the snare technique described above or by cutting the growth with a knife. Further, the growth can be mechanically lifted after the injection to further facilitate removal. Saline injection into the submucosa underneath and surrounding sessile polyps mechanically compresses blood vessels and the epinephrine causes vasoconstriction. In addition, the submucosa is expanded separating the underlying tissue from the mucosa. This increased space provides a “cushion” in preventing thermal, transmural injury to the underlying tissues. Further, some extensive flat polyps, after being elevated by this injection, will be endoscopically resectable when they were not resectable without the injection technique. However, this technique has drawbacks. The “elevated” site is easily deformable upon compression for resection. Further, the elevation dissipates rapidly over time and disappears, requiring repeated injections of saline. Other materials, such as a hypertonic, a 50% glucose solution, or a synthetic ocular lubricant provides a more persistent elevation, but can cause more injuries and increases the perforation risk in the intestinal region due to the small wall thickness. Still further, even with slower absorbing solutions, the mere resection of the tissue exposes the diffused solution to the surface and creates “leaks” in the tissue, sometimes hindering completion of the resection.

As a further alternative, difficult or larger growths to be resected by piecemeal resection through multiple injections of saline for a localized elevation and removal and then repeated in different areas as needed. In some case, the injection is unnecessary but is performed for safety reasons and because of the constraints of snare size. However, such removal creates certain difficulties for the endoscopist and the pathologist. There is often considerable debris and charring at the polypectomy site, making it difficult to assess the completeness of the excision. This probably explains the variable rates of recurrence; rates as high as 48% have been reported at follow-up due to remnant adenoma tissue. Fragmentation and diathermy artifact also make pathological interpretation and evaluation of resection margins difficult.

Another proposed procedure is to use a multichannel endoscope and insert a balloon into the submucosal tissue at a particular location through one channel of the endoscope. The balloon is inflated through the endoscope to enlarge the tissue and cause greater protrusion and access. Another channel in the endoscope is used to insert the knife and resect the tissue. The balloon is then deflated and moved to another location until the procedure is accomplished through the endoscope. The disadvantage is that the balloon acts as a “tether” to the endoscope, prohibiting movement of the endoscope to surrounding tissue areas that may need resection, especially in larger growths. Thus, the balloon has to be deflated, and the endoscope relocated, and the balloon reinflated for each portion of the resection. The system also appears limited to a multichannel endoscope to function.

Thus, for the state of the art, there are no reliable procedures and instrument(s) for the removal of lesion and other undesirable growths particularly those larger than roughly 2 cm in diameter that can be used to successfully 1) present the larger mucosal and submucosal growth to the surgeon that allows for 2) sufficient time to complete the removal, and 3) ensure sufficient margin around an intact cancerous tissue resection. Therefore, innovative procedures, materials, and tools are needed to expand the efficacy and scope of endoscopic removal of larger gastrointestinal lesions and other unwanted growths.

### BRIEF SUMMARY

The present disclosure provides a method, apparatus, and system to facilitate endoscopic insertion of a balloon into tissues, such as submucosal tissues, and optional resection of tissues elevated by the balloon when inflated. The system includes an endoscope, such as a single channel endoscope, with a delivery system for a detachable balloon having a seal. The delivery system includes a conduit suitable for delivering fluid such as gas or liquid to the balloon. The system also includes a knife suitable for resecting at least a portion of tissue that is elevated when the balloon is inflated. The balloon can be inserted under the tissue, such as a submucosal insertion, and inflated. The balloon can then be untethered from the delivery system and deployed submucosally while the endoscope is manipulated around the affected area and the affected tissue knife resected. The endoscope can be retracted from the area. The balloon can be made of biodegradable material that can dissolve or be absorbed over a period of time, can be retractable, or can remain in position for other purposes. The system and method can be applied to other areas and medical procedures, such as insertion into the gastrointestinal tract for esophageal acid reflux control, bladder and incontinence control, and other applications that require insertion of a balloon for a period of time, independently or in combination with removal of tissue. The system and method can also be applied to other therapeutic interventions and diagnostic strategies for disease identification and treatment in other organs.

The disclosure provides a system for endoscopically inserting a balloon into a layer of tissue, comprising: an endoscope having a least one channel; a balloon delivery system having a catheter with a fluid channel and a balloon on a distal portion of the catheter, the balloon being detachable from the catheter after inflation and adapted to remain inflated after detachment.

The disclosure also provides a method of endoscopically causing a tissue to protrude, comprising: inserting an endoscope through an opening in a body to a tissue; inserting a balloon delivery system having a balloon through the endoscope at least partially under the tissue; inflating the balloon to cause the tissue to protrude relative to a position of the tissue existing prior to inserting the endoscope; detaching the balloon from the balloon delivery system while the balloon is inflated; and allowing the inflated balloon to remain at least partially under the tissue for a period of time.

### BRIEF DESCRIPTION OF THE DRAWINGS

While the concepts provided herein are susceptible to various modifications and alternative forms, only a few specific embodiments have been shown by way of example in the drawings and are described in detail below. The figures and detailed descriptions of these specific embodiments are not intended to limit the breadth or scope of the concepts or the appended claims in any manner. Rather, the figures and detailed written descriptions are provided to illustrate the concepts to a person of ordinary skill in the art as required by 35 U.S.C. § 112.

FIG. 1 is a schematic cross sectional view of tissues with growths.

FIG. 2 is a schematic view of an exemplary embodiment of an endoscope system according to the invention.

FIG. 2A is a schematic cross-sectional enlarged view of portions of the exemplary endoscope system of FIG. 2.

FIG. 3 is a cross-sectional schematic view of a portion of a tissue having an unwanted growth thereon.

FIG. 4 is a cross-sectional schematic view of a catheter inserted into submucosal tissue.

FIG. 5 is a cross-sectional view of a zone created by the injection of fluid into submucosal tissue.

FIG. 6 is a cross-sectional schematic view illustrating a balloon inserted into the expanded zone in the submucosal tissue caused by the fluid injection.

FIG. 7 is a cross-sectional schematic view showing an inflated balloon in the submucosal tissue.

FIG. 8 is a cross-sectional schematic view of the balloon detached from the endoscope, allowing the endoscope to be manipulated around the growth.

FIG. 9 is a cross-sectional schematic view showing a removed growth with the balloon in the submucosal tissue.

FIG. 10 is a schematic flow chart illustrating an exemplary procedure.

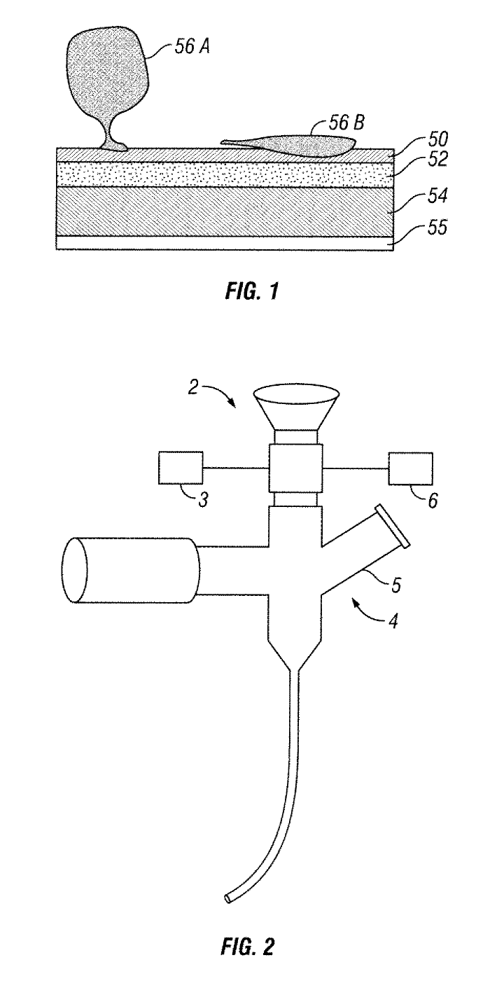

### DETAILED DESCRIPTION

One or more illustrative embodiments of the concepts disclosed herein are presented below. Not all features of an actual implementation are described or shown in this application for the sake of clarity. It is understood that the development of an actual embodiment, numerous implementation-specific decisions must be made to achieve the developer's goals, such as compliance with system-related, business-related and other constraints, which vary by implementation and from time to time. While a developer's efforts might be complex and time-consuming, such efforts would be, nevertheless, a routine undertaking for those of ordinary skill in the art having benefit of this disclosure.

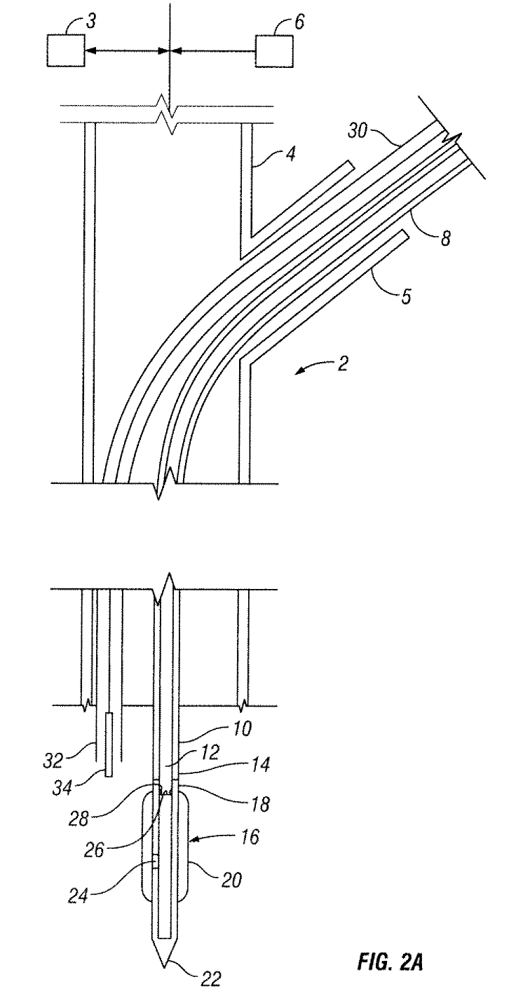

FIG. 2 is a schematic view of an exemplary embodiment of an endoscope system according to the invention. FIG. 2A is a schematic cross-sectional enlarged view of portions of the exemplary endoscope system of FIG. 2. The figures will be described in conjunction with each other. The endoscope system 2 generally includes an endoscope assembly 4, as is known to those with ordinary skill in the art. An endoscope assembly 4 of varying sizes can be inserted through portions of a body to illuminate and remotely view portions of the body through which the endoscope assembly is inserted. A side injection port 5 is used to insert various catheters, tools, and other assemblies with the endoscope assembly 4. The endoscope assembly 4 can be a single channel endoscope or a multi-channel endoscope having two or more channels. Most practitioners use single-channel endoscopes due to their simplicity and lower cost. Thus, one embodiment of the present disclosure uses a single channel endoscope. Other embodiments can use multi-channel endoscopes as desired. The term “endoscope” is used broadly in this application and includes any tool insertable into a body having a channel through which tools and other devises can be placed and used, whether inserted through a natural body orifice or through an artificially created opening, such as through an incision or other procedure, and thus includes a laparoscope modified to be able to convey tools and related instruments for purposes herein. When a distal lens is unavailable, known imaging techniques external to the body can be used for locating the tools internal to the body.

The endoscope system 2 can further include a balloon delivery system 8. The balloon delivery system 8 generally includes a conduit 10 having a fluid channel 12, and a detachable element 14 to deploy the balloon at the location, allowing the endoscope to be manipulated to other locations as necessary. The balloon delivery system 8 can further include a balloon assembly 16. The detachable element 14 can be used to couple the conduit 12 to the balloon assembly 16. In at least one embodiment, the detachable element 14 can include a latch to hold the balloon on the endoscope during insertion and inflation. A slide or release mechanism can be operated by the practitioner to push off the balloon and/or release the balloon from the endoscope at the appropriate location. For example, a similar latching assembly sold by Boston Scientific is presently used for releasing surgical clips after actuation with an endoscope, and such teaching can be adapted by one with ordinary skill in the art to the structure and particular needs of the present invention, given the teachings and guidance provided herein.

In at least one embodiment, the balloon assembly 16 generally includes a balloon and a balloon seal 26 for sealing the balloon after inflation and deployment. The balloon assembly can include a connection conduit 18, disposed adjacent to the detachable element 14 and a balloon 20 coupled to the connection conduit 18. The end of the balloon assembly 16 can include a tip 22. In at least one embodiment, the tip 22 is closed to allow fluid in the balloon assembly 16 to become pressurized and flow through an opening 24 in the connection conduit 18 to inflate the balloon 20.

The seal 26 can be self-sealing, such as a check valve, dome valve, and self-sealing septum. The check valve can include such valves as read valves, flapper valves, and other self-sealing valves. In at least one embodiment, the seal 26 can seal against a seat 28 within the balloon assembly 16.

The balloon assembly can be guided along a guide wire to a planned location, as described herein. In some embodiments, the guide wire will be external to the balloon assembly. In other embodiments, the guide wire can be internal to the balloon assembly and the balloon assembly can be modified to allow the guide wire to pass therethrough.

The balloon can be made of medical grade plastic. In some embodiments, the balloon can be made of biodegradable material such as without limitation, polylactic acid materials, glycolides, and other materials that may polymerize and degrade with time in the body. Such materials can include biodegradable polymers including, but not limited to, glycolic acid-based polymers, such as polyglycolide (PGA); lactic acid-based polymers, such as polylactide (PLA); polyanhydrides; polyorthoesters; polyphosphazenes; poly(dioxanone) copolymers; poly(trimethylene carbonate) copolymers; poly(e-caprolactone) homopolymers and copolymers; LPLA; DLPLA; PCL; PDO; PGA-TMC; DLPLG; and polyhydroxybutyrate (PHB) and polyhydroxyvalerate (PHV) polymers and copolymers, such as Biopol(r) (Monsanto Co., St. Louis, Mo.). In other embodiments, the balloon can be made of non-biodegradable material, for example, if the material is to remain intact for an extended period of time. The filling agent of the balloon can be any medically safe material including liquid saline, dextrose solutions, water, gases, such as air or nitrogen, and other fluids. Further, the fluid can be a hardenable material, known to those in the art. The balloon can be provided in different sizes and shapes depending on the size of the tissue to be elevated. Further, the term “balloon” is used broadly to encompass one or more balloons. For example, multiple balloons can be placed in position instead of one larger balloon as may be appropriate for the particular growth to be removed. Further, the balloon can include radio opaque material.

An additional tool that can be inserted through the side injection port is a knife sheath 30. The knife sheath 30 generally includes a tip 32 where a retractable knife 34 can extend to cut or abrade various tissues after being guided to the proper location. If the sizes allow, both the knife sheath 30 and the balloon delivery system 8 can be inserted through the injection port at the same time. In some embodiments, insufficient room is available and the balloon delivery system 8 and the knife sheath 30 can be sequentially inserted through the injection port. Advantageously, the balloon delivery system can leave the balloon assembly in location, such as in a submucosal tissue, and be removed from the endoscope assembly 4. Then, the knife sheath 30 can be inserted through the injection port for resection of the unwanted growth. This aspect alone remarkably differs from known methods and systems in that the knife sheath 30 of the present disclosure is not tethered to a fixed location with the balloon.

The endoscope assembly 4 generally includes a monitor and lens system 3 for viewing the distal end of the endoscope as it is inserted into the body and guided to the desired location. Further, the endoscope assembly 4 includes a light and power source 6 for providing illumination to the endoscope to view the relevant tissues.

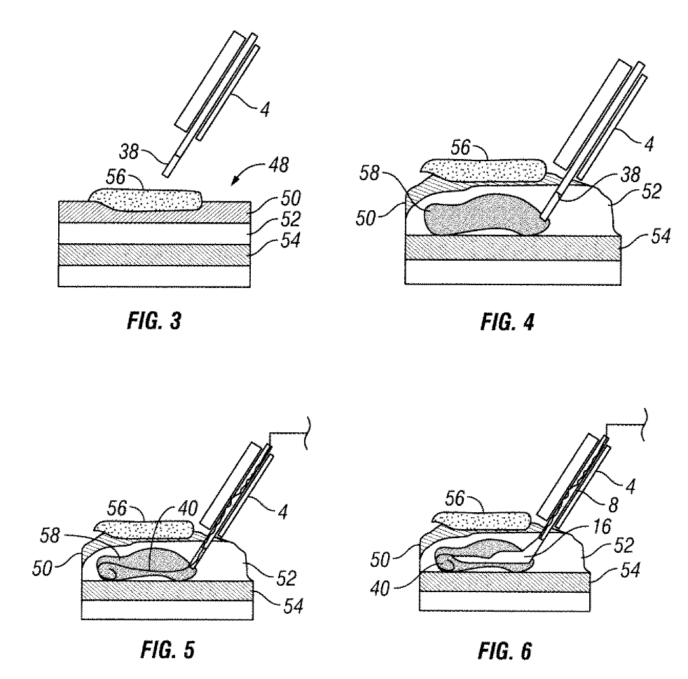

FIGS. 3 through 9 illustrate at least one series of procedures that can be performed using the endoscope system 2 described above with the endoscope assembly 4 and the balloon delivery system 8 and balloon assembly 16. The illustrations are schematic only and are representative of only one possibility of using the system. Other steps and methods could be adapted by one with ordinary skill in the art given the disclosure contained herein.

The disclosed balloon assembly and related systems and methods are exemplary and other and further assemblies and systems can be used and are contemplated. For example and without limitation, other exemplary balloon delivery assemblies and systems include those devices shown in U.S. Pat. Nos. 4,441,495, 5,188,558, 5,304,123, 6,736,793, 6,312,405, and 6,666,828 and US Publication 20060079923, having detachable and sealable balloons. The teaching of such disclosures are incorporated herein by reference.

Further, variations on the use of the guide wire, including a guide wire external to balloon or internal to the balloon, no guide wire, guide wire at different steps, retracting the guide wire at different steps, and other variations are possible. Such variations are contemplated and are intended to be included within the scope of the claims contained herein.

FIG. 3 is a cross-sectional schematic view of a portion of a tissue having an unwanted growth thereon. The portion of tissue 48 generally includes a mucosal tissue 50, a submucosal tissue 52 below the mucosal tissue, and a muscularis tissue 54 below the submucosal tissue. An unwanted growth 56 can occur on the mucosal tissue 50. Generally, the growth can be a polyp or other tissue that may have a variety of sizes and shapes including a relatively flat surface. The growth can also be a portion of tissue to be stripped during a medical procedure, such as vascular surgery for retrieval of vein grafts. Rounded growths with a head and a stalk can be generally removed through snares and other endoscopic tools known to those with ordinary skill in the art. Flattened growths are more problematic due to accessibility of a larger surface that follows the shape of the underlying tissue. Other types of growths can have similar difficulties in removal or resection. While a variety of growths can benefit from the present disclosure, it is envisioned that the more difficult growths to resect may especially benefit from the present disclosure. Thus, for illustrative purposes, the more flattened growth 56 will be described in the resection procedure, although it is to be understood that the procedure could apply to other types of growths.

FIG. 4 is a cross-sectional schematic view of an endoscope inserted into submucosal tissue. The endoscope system 10 can be guided through a body cavity such as an esophagus or colon to an appropriate location. A fluid injection catheter 38 with a needle can be disposed down the endoscope to the submucosal tissue so that fluid can be injected into the submucosal tissue 52. This fluid can include saline and other solutions. In some embodiments, the saline can be dyed with methylene blue. Another liquid besides saline is D50 (50% w/v dextrose). D50 can remain in a concentrated area of the submucosal tissue longer than saline.

FIG. 5 is a cross-sectional view of a zone created by the injection of fluid into submucosal tissue. The zone 58 raises or protrudes the growth 56 to a higher elevation relative to surrounding tissue. The higher elevation assists in access and resection of the growth 56 from the mucosal tissue 50. Additional injections can be made at different points through the tissue to create a noncircular, flattened, or nonuniform shaped zone 58 depending upon the size, extent, and shape of the growth 56. The needle and injection catheter can be removed and a guide wire 40 can be inserted into the endoscope 2 and guided into the zone 58. The guide wire can provide guidance for various tools, balloons, and other instruments inserted during the procedure.

FIG. 6 is a cross-sectional schematic view illustrating a balloon inserted into the expanded zone in the submucosal tissue caused by the fluid injection. A balloon assembly 16 can be inserted through the endoscope system 2 with the balloon delivery system 8. In at least one embodiment, the balloon assembly can be inserted along the guide wire 40. In some embodiments, the balloon can be inserted independent of the guide wire and thus the illustrated guide wire is not to be exclusive of other options. Further, the balloon can include different sizes and shapes as may be appropriate. The determination of the balloon size and shape can be made after endoscopic review of the growth 56 during the procedure. In some embodiments, the balloon can be inserted directly into the submucosal tissue 52 without the injection of fluid to create a zone 58. Other embodiments of balloons and balloon assemblies are possible and contemplated.

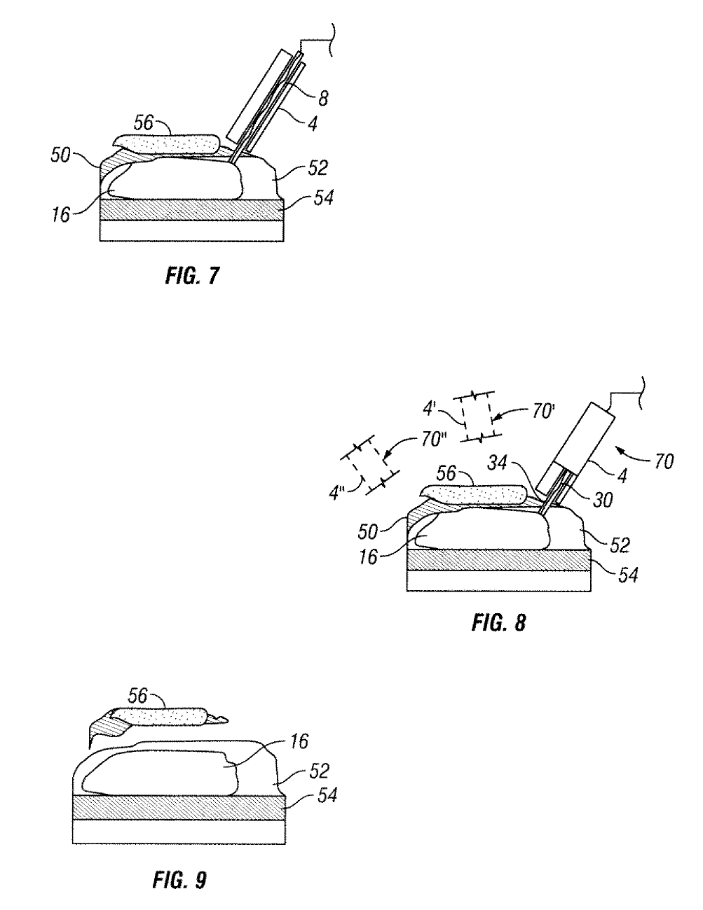

FIG. 7 is a cross-sectional schematic view showing an inflated balloon in the submucosal tissue. The balloon delivery system 8 can inflate the balloon assembly 16 when positioned in the selected location. The balloon assembly 16 can retain the zone created by the solution injected into the submucosal tissue. In at least aspect, the balloon can retain the protrusion of the growth 56.

FIG. 8 is a cross-sectional schematic view of the balloon detached from the endoscope. The balloon assembly 16 having the inflated balloon can be detached from the endoscope assembly 4 and the balloon delivery system 8, shown in FIG. 7. In at least one embodiment, the balloon delivery system 8 can be retracted from the endoscope assembly. In other embodiments, with sufficient room, the balloon delivery system 8 can remain down the endoscope system while other procedures are performed. Further, the balloon delivery system can be retracted and reinserted with one or more other balloon assemblies to be placed in zone 58 or other locations.

Generally, it will be desirable to remove the growth 56. Thus, a knife sheath 30 with a knife 34 can be inserted down the endoscope assembly to remove the growth 56. Advantageously, the endoscope is not tethered to the balloon assembly 16, so that the endoscope can be moved from the first position 70 to a second position 70′ to a third position 70″ as appropriate to further resect portions of the growth 56. The term “knife” is used broadly to include any method of removing material including ablation methods, such as with an argon plasma coagulator, and other ablation tools, high frequency knives, grinders, blades, snares, and other tissue removing devices.

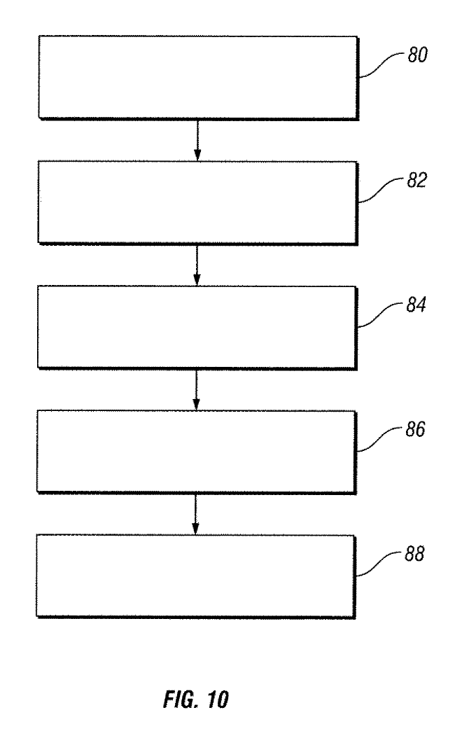

FIG. 9 is a cross-sectional schematic view showing a removed growth with the balloon in the submucosal tissue. The growth 56 can be removed as a single piece or in multiple pieces in different stages as may be appropriate. In at least some embodiments, the balloon can remain in place in the submucosal layer. In such instances, it may be advantageous to make the balloon assembly out of biodegradable material. In other embodiments, the balloon can be retrieved through the endoscope using various tools, such as forceps and other devices. It may be advantageous to puncture or otherwise deflate the balloon assembly prior to removal so that the fluid contained therein can be allowed to escape and collapse the balloon for easier retrieval.

In other embodiments, it may be advantageous to simply leave the balloon in position on a more long-term basis. Such instances could include applications where it is desired to maintain a protruding portion of the mucosal tissue 50. Further, in such more permanent, applications, the balloon assembly could be used for other types of gastrointestinal applications for incontinence, acid reflux, and so forth. In such applications, the procedure may be directed primarily to inserting a balloon and detaching the balloon from the balloon delivery system without necessarily resecting tissue. As one exemplary and nonlimiting application, the balloon could be asserted into an esophageal submucosal tissue above the stomach to restrict the size of the esophagus and reduce acid reflux damage in the esophagus. The present disclosure uniquely offers a detachable balloon that can be inserted submucosally and left in position in an inflated condition using an endoscopic procedure to access areas of the body not generally accessed.

The concepts disclosed herein using a detachable balloon inserted endoscopically can also be used not only in surgical situations that require separation of cell layers like endometrial stripping but also for therapeutic procedures, such as procedures where there is use for contained bulking agents such as gastroesophageal reflux disease (GERD), acid reflux, bowel and urinary incontinence and vesicoureteral reflux, subcutaneous dissection, tamponade in closed spaces, a lithotripsy target, subfacial, submusclar dissection, or as a prosthesis. A detachable balloon that can be deployed in situ can assist in subcutaneous dissection for plastic and reconstructive surgery, and vascular surgery for retrieval of vein grafts. An endoscopically detachable balloon can also provide intervertebral disc prosthesis or a joint cartilage or fluid prosthesis. As another example, in lithotripsy, a balloon can be deployed above or below a stone in the bile duct, ureter, or bladder with radioopaque contrast for the lithotripsy technician to aim and destroy the radiolucent stones therebetween. Further, a deployed balloon with radioopaque contrast can provide for focused radiotherapy of tumors by marking the tumor above and below with a balloon in the esophagus, stomach, pancreas, colon, bronchus, kidney, bladder, and other body tissue and organs. Furthermore, the deployed balloon can be designed such that it contains therapeutic ingredients like drugs, cells, peptides or enzymes under the mucosa and can be left in situ to provide local or regional therapeutic effects. For example, it can be conceived that following surgical resection of cancerous tissue, a balloon containing chemotherapeutic agent is deployed and left close to the resection site, so that there is localized delivery to prevent recurrence. Such a balloon can also be constructed with a slow-release material and be filled with antimetastatis and/or anti-angiogenesis agents; this kind of delivery will greatly aid the release of larger doses of therapeutic agents more effectively, since the deployed balloon is close to the site of disease. This kind of delivery reduces the systemic effects and will also help deliver large doses of therapeutic agents more effectively.

FIG. 10 is a flow chart illustrating one non-limiting example that is similar to the procedures that have been described above. Step 80 includes passing an endoscope into the UGI tract or colon. Step 82 includes injecting a saline solution with an injection catheter into the submucosal tissue to create a “space” and lift a lesion. Step 84 includes inserting a balloon catheter into the submucosal saline space. Step 86 includes inflating a balloon in the submucosal tissue, detaching the balloon, and removing the catheter. Step 88 includes inserting a needle knife to cut on the periphery of the elevation until the lesion is removed.

The invention has been described in the context of various embodiments and not every embodiment of the invention has been described. For example, the mucosal tissues have been described, but it is contemplated and understood that the invention may be used on other body tissues and such use is included within the scope of the claims. Apparent modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, the Applicant intends to protect all such modifications and improvements to the full extent that such falls within the scope or range of equivalent of the following claims.

The various methods and embodiments of the invention can be included in combination with each other to produce variations of the disclosed methods and embodiments, as would be understood by those with ordinary skill in the art, given the understanding provided herein. Also, various aspects of the embodiments could be used in conjunction with each other to accomplish the understood goals of the invention. Also, the directions such as “top,” “bottom,” “left,” “right,” “upper,” “lower,” and other directions and orientations are described herein for clarity in reference to the figures and are not to be limiting of the actual device or system or use of the device or system. Unless the context requires otherwise, the word “comprise” or variations such as “comprises” or “comprising”, should be understood to imply the inclusion of at least the stated element or step or group of elements or steps or equivalents thereof, and not the exclusion of a greater numerical quantity or any other element or step or group of elements or steps or equivalents thereof. The device or system may be used in a number of directions and orientations. Further, the order of steps can occur in a variety of sequences unless otherwise specifically limited. The various steps described herein can be combined with other steps, interlineated with the stated steps, and/or split into multiple steps. The term “coupled,” “coupling,” “coupler,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, directly or indirectly with intermediate elements, one or more pieces of members together and can further include without limitation integrally forming one functional member with another in a unity fashion. The coupling can occur in any direction, including rotationally. Additionally, the headings herein are for the convenience of the reader and are not intended to limit the scope of the invention.

Further, any references mentioned in the application for this patent as well as all references listed in the information disclosure originally filed with the application are hereby incorporated by reference in their entirety to the extent such may be deemed essential to support the enabling of the invention. However, to the extent statements might be considered inconsistent with the patenting of the invention, such statements are expressly not meant to be considered as made by the Applicant.

1. A system for endoscopically inserting a balloon into a layer of tissue, comprising:
an endoscope having a least one channel;
a balloon delivery system having a catheter with a fluid channel and a balloon on a distal portion of the catheter, the balloon being detachable from the catheter after inflation and adapted to remain inflated after detachment.

2. The system of claim 1, wherein balloon comprises a balloon seal sealable after detachment from the catheter of the balloon delivery system.

3. The system of claim 2, wherein the seal comprises a self-sealing valve.

4. The system of claim 1, further comprising a knife adapted to be inserted through the endoscope.

5. The system of claim 1, further comprising a fluid injection catheter adapted to be inserted through the endoscope.

6. The system of claim 5, wherein the catheter of the balloon delivery system comprises the fluid injection catheter.

7. The system of claim 1, wherein endoscope comprises a single channel endoscope.

8. The system of claim 1, wherein the balloon delivery system is adapted to deliver different sizes of balloons.

9. The system of claim 1, further comprising a filling material for the balloon, the filling material being a fluid.

10. The system of claim 9, wherein the filling material comprises a fluid adapted to harden after inflation of the balloon.

11. The system of claim 1, wherein the balloon comprises a material adapted to degrade and be absorbed or eliminated from the tissue.

12. The system of claim 1, wherein endoscope comprises a multi-channel endoscope.

13. A method of endoscopically causing a tissue to protrude, comprising:
inserting an endoscope through an opening in a body to a tissue;
inserting a balloon delivery system having a balloon at least partially under the tissue;
inflating the balloon to cause the tissue to protrude relative to a position of the tissue existing prior to inserting the balloon;
detaching the balloon from the balloon delivery system while the balloon is inflated;
allowing the inflated balloon to remain at least partially under the tissue for a period of time.

14. The method of claim 13, further comprising injecting fluid under the tissue.

15. The method of claim 14, further comprising expanding the tissue through the fluid injection prior to inserting the balloon.

16. The method of claim 15, further comprising inserting a guide wire through the endoscope into the tissue to provide a guide path for inserting the balloon.

17. The method of claim 13, further comprising resecting at least a portion of the protruding tissue.

18. The method of claim 13, further comprising relocating the endoscope around the protruding tissue to resect portions of the tissue while the balloon remains disposed in the tissue.

19. The method of claim 13, further comprising cutting a portion of the tissue and inserting the balloon under the tissue independent of a guide wire.

20. The method of claim 13, wherein detaching the balloon from the balloon delivery system further comprising automatically sealing the balloon.

21. The method of claim 13, allowing the balloon to degrade in position during inflation after a period of time.
